(19) United States
(12) Patent Application Publication
(10) Pub. No.: US 2024/0325524 A1
(43) Pub. Date: Oct. 3, 2024

(54) COMPOSITIONS AND METHODS FOR INDUCING IMMUNE TOLERANCE

(71) Applicant: THE REGENTS OF THE UNIVERSITY OF MICHIGAN, Ann Arbor, MI (US)

(72) Inventors: Nicholas W. Lukacs, Brighton, MI (US); Srikanth Elesela, Ann Arbor, MI (US); Simon Hogan, Ann Arbor, MI (US)

(21) Appl. No.: 18/697,529

(22) PCT Filed: Oct. 6, 2022
(86) PCT No.: PCT/US2022/045853

§ 371 Date: Apr. 1, 2024

Related U.S. Application Data
(60) Provisional application No. 63/253,431, filed on Oct. 7, 2021.
(60) Provisional application No. 63/288,915, filed on Dec. 13, 2021.

(51) Int. Cl. A61K 39/35; A61K 39/00; A61K 39/395; A61P 37/08

(52) U.S. Cl. CPC A61K 39/35; A61K 39/39583; A61P 37/08; A61K 2039/541; A61K 2039/542

(57) ABSTRACT

The disclosure is directed compositions comprising antigen-specific IgA immune complexes and methods of using the compositions to induce immune tolerance against allergens in a subject who has an established Th2 polarized immune response at one or more mucosal sites prior to administering the composition.

### FIELD

The present disclosure relates to compositions comprising antigen-specific IgA immune complexes and methods of using the compositions to induce immune tolerance against allergens.

### BACKGROUND

The rate of allergic diseases, such as atopic dermatitis, asthma, and food allergies, has increased considerably in the past three decades, especially in infants and young children in Western countries. The increased prevalence of allergies have been attributed to several variables, including mode of birth, breast feeding, and exposure to the proper environment to allow appropriate assembly of the microbiome. Severe food allergy-related reactions, also known as food-triggered anaphylaxis, are serious life threatening reactions responsible for 30,000-120,000 emergency department visits, 2,000-3,000 hospitalizations, and approximately 150 deaths per year in the United States (Sampson et al., Pediatrics, 111:1601-1608 (2003); and Ross et al., J. Allergy Clin. Immunol., 121:166-171 (2008)). The onset of symptoms are variable, occurring within seconds to a few hours following exposure to the dietary allergen, and multiple organ systems are often affected, including gastrointestinal (GI), cutaneous, respiratory, and cardiovascular (Wang et al., Clin. Exp. Allergy, 37:651-660 (2007)). Cutaneous symptoms (e.g., urticaria and angioedema) are the most common, occurring in approximately 80% of cases. GI symptoms occur in as much as 40% of cases, including cramping, abdominal pain, nausea, emesis, and diarrhea (Sampson et al., The New England Journal of Medicine, 327:380-384 (1992)). Recent clinical data suggest a link between GI manifestations and more severe anaphylactic phenotypes, including hypotension and hypoxia (Schrander et al., J. Pediatr. Gastroenterol. Nutr., 10:189-192 (1990); Troncone et al., Allergy, 49:142-146 (1994); Van Elburg et al., Pediatr Allergy Immunol, 4:79-85 (1993); Calvani et al., Pediatric Allergy and Immunology, 22:813-819 LID-810.1111/j. 1399-3038.2011.01200.x [doi] (2011); and Brown, S. G. A., J Allergy Clin Immunol, 114:371-376 (2004)).

IgA is the most abundant immunoglobulin and is found predominantly at mucosal surfaces. Its functions appear to be associated with binding to pathogens to aggregate and immobilize pathogenic microbes and substances in mucosal tissue to block colonization and penetration into the underlying tissue, including in the lung and intestinal tract. A number of studies have linked IgA levels with the development of modified or tolerogenic immune responses. For example, the total dose of IgA in colostrum has been shown to provide early protection against infectious organisms and is inversely related to development of atopic dermatitis in the first two years of life (Orivuri, clin exp allergy, 2014). Breast milk also contains TGFβ1, which promotes IgA class switching in B cells and reduces inflammatory immune responses. Finally, supplementation with specific bacteria, such as Lactobacilli, has been shown to induce increased levels of total and specific IgA in the mucosal tissue through interactions with dendritic cells and production of retinoic acid, leading to decreased allergic responses (Mikulic et al., Cell Mol Immunol., 14 (6): 546-556 (2017); and Prescott et al., Clin Exp Allergy, 38 (10): 1606-14 (2008)).

There remains a need for methods and compositions for treating allergies, such as food allergies.

### BRIEF SUMMARY

The disclosure provides use of a composition comprising a pharmaceutically acceptable carrier and an immune complex comprising an allergen bound to an IgA immunoglobulin, or antigen-binding fragment thereof, for inhibiting an allergic reaction to a food allergen in a subject, wherein the IgA immunoglobulin is specific for the allergen. The composition may be formulated for one or more routes of administration including local and systemic delivery. Exemplary formulations include formulations for pulmonary (e.g., inhalation) administration as well as formulations for oral administration.

The disclosure also provides a method of inhibiting an allergic reaction in a subject, which method comprises administering to the subject a composition comprising an immune complex and a pharmaceutically acceptable carrier, wherein the immune complex comprises an allergen bound to an IgA immunoglobulin specific for the allergen. Administering a composition comprising an immune complex of the disclosure may be used to prophylactically treat a subject in order to prevent and/or reduce an allergic immune response (e.g., prevent and/or reduce a Th2 type immune response) in the subject. Administering a composition comprising an immune complex of the disclosure may also be used to therapeutically treat a subject in order to ameliorate and/or reduce an allergic immune response (e.g., a Th2 type immune response at one or more mucosal sites) in the subject. The disclosure is not limited by the route or means of administrating the composition comprising the immune complex. Examples of routes of administration include but are not limited to pulmonary administration (e.g., via inhalation), enteral administration (e.g., via oral, gastric or duodenal (e.g., feeding tube), and/or rectal administration) and other routes described herein.

### BRIEF DESCRIPTION OF THE DRAWING(S)

FIG. 1 depicts (A) an exemplary administration regimen of IgA immune complex to allergic animals into the airway mucosa during systemic sensitization that: (B and C) protects animals from mucus hypersecretion, (D) alters immune cytokine responses, and (E) increases total number of Treg cells.

FIG. 2 depicts (A) an exemplary administration regimen of IgA immune complex into the airway mucosa during sensitization protects animals against development of food allergy upon challenge that: (B) blocks clinically relevant diarrhea and temperature decreases indicative of anaphylaxis, (C) reduces systemic IgE and evidence of mast cell degranulation (mMCPt1), (D) reduces Th2 cytokines while increasing the suppressive cytokine IL-10. The lack of effect with a different allergen, peanut (PE), demonstrates allergen specificity for the responses.

FIG. 3 depicts (A) a modified food allergy model and that transfer of Th2 skewed cells from TCR-transgenic DO11 Balb/c mice do not develop into Treg cells, that the IgA Immune complex induces (B) significant clinical alterations and (C) significant cytokine alterations, and (D) that the reduction in IL-4+ Transferred DO11 Th2 skewed cells were not overlapping with the Foxp3+ Treg cells that were developed during the tolerizing responses, indicating that the induction of the Treg cells that are associated with the tolerizing effect of immune complex are generated from non-Th2 skewed cells.

FIG. 4 depicts (A) the IgA immune complex alters the accumulation of mast cells, (B) reduces IgE and mMCPT-1, and (C) promotes a tolerogenic environment in the intestine of food allergic mice, even when highly skewed allergen-specific Th2 cells are transferred into sensitized mice.

FIG. 5 depicts (A) Incidence of diarrhea and decreased temperature as clinical indicators of anaphylaxis were regulated only in the IgA-TNP-ova immune complex treated animals. (B) Measurement of serum IgE and Mcpt indicated decreased levels of both mast cell activator and product were observed. (C) Increased Treg cells in mesenteric lymph nodes were highly significant in the oral IgA immune complex treated animals. Data represent the mean±SE from 5-6 mice/group. *P<0.05, **P<0.01, ***P<0.005.

FIG. 6 depicts isolated mRNA assessed for (A) TGFb or (B) IL-10 by qPCR analysis. (C) In separate studies, BMDC were incubated with IgA immune complex or appropriate controls and after an overnight incubation the cells were washed and combined with naïve splenic CD4+ T cells from DO11 IL-4-GFP reporter mice that are TCR transgenic for ovalbumin and incubated for 48 hrs in the presence of ovalbumin without TNP. Data represent mean±SE from 3 repeat experiments. *P<0.05, **P<0.01, ***P<0.005.

### DEFINITIONS

To facilitate an understanding of the present technology, a number of terms and phrases are defined below. Additional definitions are set forth throughout the detailed description.

The term “allergy,” as used herein, refers to a chronic condition involving an abnormal or pathological immune reaction to a substance (i.e., an “allergen”) that is ordinarily harmless in normal/healthy individuals. Allergy is one of a class of immune system responses that are termed hypersensitivity reactions. The terms “hypersensitivity” and “hypersensitivity reactions,” as used herein, refer to harmful immune responses that produce tissue injury and may cause serious disease. Hypersensitivity reactions have been classified into four types, and allergy is often equated with type I hypersensitivity (immediate-type hypersensitivity reactions mediated by IgE). An “allergen” refers to any substance (e.g., an antigen) that induces an allergic reaction in a subject. Examples of allergens include, but are not limited to, aeroallergens (e.g., dust mite, mold, spores, plant pollens such as tree, weed, and grass pollens), food products (milk, egg, soy, wheat, nut, or fish proteins), animal products (e.g., cat or dog hair), drugs (e.g., penicillin), insect venom, and latex. The term “allergic reaction,” as used herein, refers to a pathological reaction of the immune system triggered by the exposure of an individual to a foreign, and typically harmless, substance. Thus, “inhibiting” an allergic reaction refers to the suppression, amelioration, or prevention of a pathological reaction to an allergen (e.g., by redirecting Th2-polarized immune responses in a subject (e.g., in a subject with Th2 mediated disease) toward a Th1-type immune response (e.g., skew toward a Th1 type immune response and/or generate a more balanced Th1/Th2 type immune response)).

The terms “peptide,” “polypeptide,” and “protein” are used interchangeably herein, and refer to a polymeric form of amino acids comprising at least two or more contiguous amino acids, which can include coded and non-coded amino acids, chemically or biochemically modified or derivatized amino acids, and polypeptides having modified peptide backbones.

As used herein, the terms “treatment,” “treating,” and the like, refer to obtaining a desired pharmacologic and/or physiologic effect. Preferably, the effect is therapeutic, i.e., the effect partially or completely alleviates or cures an injury, disease, or condition and/or an adverse symptom attributable to the injury, disease, or condition. Similarly, a “therapeutic agent,” is any substance, molecule, or compound that is capable of alleviating or curing an injury, disease, or condition and/or adverse symptom when administered to a subject in need thereof. To this end, the methods described herein desirably comprise administering a “therapeutically effective amount” of an immune complex comprising IgA. A “therapeutically effective amount” refers to an amount effective, at dosages and for periods of time necessary, to achieve a desired therapeutic result (e.g., inhibiting an allergic reaction). The therapeutically effective amount may vary according to factors such as the disease or condition severity, age, sex, and weight of the individual, and the ability of therapeutic agent to elicit a desired response in the individual.

As used herein, the terms “immunogen” and “antigen” refer to an agent (e.g., an allergen or a microorganism (e.g., bacterium, virus or fungus)) and/or portion or component thereof that is capable of eliciting an immune response in a subject.

The term “immunoglobulin” or “antibody,” as used herein, refers to a protein that is found in blood or other bodily fluids of vertebrates, which is used by the immune system to identify and neutralize foreign objects, such as bacteria and viruses. Typically, an immunoglobulin or antibody is a protein that comprises at least one complementarity determining region (CDR). The CDRs form the “hypervariable region” of an antibody, which is responsible for antigen binding. A whole immunoglobulin typically consists of four polypeptides: two identical copies of a heavy (H) chain polypeptide and two identical copies of a light (L) chain polypeptide. Each of the heavy chains contains one N-terminal variable (V_{H}) region and three C-terminal constant (CH1, CH2, and CH3) regions, and each light chain contains one N-terminal variable (V_{L}) region and one C-terminal constant (C_{L}) region. The light chains of antibodies can be assigned to one of two distinct types, either kappa (κ) or lambda (2), based upon the amino acid sequences of their constant domains. In a typical antibody, each light chain is linked to a heavy chain by disulphide bonds, and the two heavy chains are linked to each other by disulphide bonds. The light chain variable region is aligned with the variable region of the heavy chain, and the light chain constant region is aligned with the first constant region of the heavy chain. The remaining constant regions of the heavy chains are aligned with each other.

The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5:511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352:624-628 (1991)); and Marks et al., J. Mol. Biol., 222:581-597 (1991)), or produced from transgenic mice carrying a fully human immunoglobulin system (see, e.g., Lonberg, Nat. Biotechnol., 23 (9): 1117-25 (2005), and Lonberg, Handb. Exp. Pharmacol., 181:69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23 (9): 1126-1129 (2005)). An antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H1} domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

As used herein, when an antibody or other entity (e.g., antigen binding domain) “specifically recognizes,” “specifically binds,” or is “specific for” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

The terms “host” or “subject,” as used herein, refer to an individual to be treated by (e.g., administered) the compositions and methods of the present invention. Subjects include, but are not limited to, mammals (e.g., murines, simians, equines, bovines, porcines, canines, felines, etc.), and most preferably includes humans. In the context of the invention, the term “subject” generally refers to an individual who is suspected of suffering from, or diagnosed as suffering from, an allergy (e.g., a food allergy).

A used herein, the term “immune response” refers to a response by the immune system of a subject. For example, immune responses include, but are not limited to, a detectable alteration (e.g., increase) in Toll-like receptor (TLR) activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system), and humoral immune responses (e.g., responses mediated by B cells (e.g., via generation and secretion of antibodies into the plasma, lymph, and/or tissue fluids)). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

The term “mucosal immunity,” as used herein, refers to the immune responses that occur at surfaces in contact with the environment, such as epidermis, gum, nose, gut, uterus and prostate. In healthy states, the mucosal immune system provides protection against pathogens but maintains a tolerance towards non-harmful commensal microbes and benign environmental substances.

The terms “immune tolerance” or “tolerance,” as used herein, refer to the state of unresponsiveness of the immune system to substances or tissues that have the potential to induce an immune response. “Central tolerance” is the primary mode by which the immune system discriminates self from non-self, and is established by deleting autoreactive lymphocyte clones before they develop into fully immunocompetent cells. Central tolerance occurs during lymphocyte development in the thymus and bone marrow for T and B lymphocytes, respectively. “Peripheral tolerance” is important for preventing over-reactivity of the immune system to various environmental factors (allergens, gut microbes, etc.), and develops after T and B cells mature and enter the peripheral tissues and lymph nodes.

Any suitable sample type, as described herein, may be obtained from any subject suspected of having an allergy. A subject (e.g., a human) is “suspected of having an allergy” if the subject is predisposed to experiencing an allergy or is exhibiting allergy symptoms. The predisposition may be genetic (e.g., a particular genetic tendency to experience the allergy), or due to other factors (e.g., environmental conditions, exposures to immunogenic compounds present in certain foods, etc.). Thus, the present invention is not to be limited to any particular risk, nor is the present invention limited to any particular allergy (e.g., any human may be susceptible to experiencing any allergy).

### DETAILED DESCRIPTION

The present disclosure is predicated, at least in part, on the discovery that IgA complexed with a cognate antigen provides immune tolerance in animals with an existing allergy response. While an understanding of a mechanism is not needed to practice the present disclosure and while the disclosure is not limited to any particular mechanism, in some aspects a composition comprising IgA complexed with antigen provides a tolerizing signal and/or generation of regulatory T cells (“Tregs” or “Treg cells”) in a subject (e.g., a subject with an existing allergic response). In some aspects, immune tolerance (e.g., via induction of Treg cells) provided by the disclosed compositions and methods prevents the progression to chronic and severe allergic responses at mucosal surfaces (e.g., the lung and gut), blocking anaphylaxis. In other aspects, immune tolerance (e.g., via induction of Treg cells) provided by the disclosed compositions and methods occurs systemically within the subject.

In some embodiments, the disclosure provides a method of inhibiting an allergic reaction in a subject, which method comprises administering to the subject a composition comprising an immune complex and a pharmaceutically acceptable carrier, wherein the immune complex comprises an allergen bound to an IgA immunoglobulin specific for the allergen, and wherein the subject has an established Th2 polarized immune response at one or more mucosal sites prior to administering the composition.

The term “immune complex (IC),” as used herein, refers to an antibody bound to a soluble antigen. An immune complex may also be referred to as an “antigen-antibody complex” or an “antigen-bound antibody.” IgA is the most abundant immunoglobulin produced and is found predominantly at mucosal surfaces. Its functions appear to be associated with binding to pathogens to aggregate and immobilize pathogenic microbes and substances in mucosal tissue to block colonization and penetration into the underlying tissue, including in the lung and intestinal tract. In addition to IgA, the mucosal surface acquires protection from pathogens via tight epithelial barriers and secretion of mucus and other substances to prevent colonization. Patients with partial or total deficiency in secretory IgA production have been shown to experience increased allergies to environmental antigens (including foods) as well as increased prevalence of autoimmune responses. These and other data suggest that IgA is involved in blocking sensitization or inducing tolerance of immune system responses at the mucosal surface. In particular, the total dose of IgA in colostrum and breast milk can provide early protection against infectious organisms, and also has been shown to be inversely related to development of atopic dermatitis in the first two years of life (Orivuri, clin exp allergy, 2014). Breast milk also contains TGFβ1, which promotes IgA class switching in B cells. Studies have also found dynamic correlations between Treg cell development, TGFβ production, IgA levels, and tolerance induction.

The immune complex of the composition may comprise a whole IgA antibody, or an antigen-binding fragment of an IgA antibody, such as any of the antibody fragments described herein. The IgA antibody, or antigen-binding fragment thereof, is specifically bound to one or more antigens. In some embodiments, the IgA immunoglobulin is specific for a single allergen, as demonstrated in the experiments described herein which indicate that the tolerizing response induced by the IgA immune complex is allergen specific. The allergen may be any suitable allergen disclosed herein or otherwise known in the art. In some embodiments, the allergen is an aeroallergen, such as dust mites, mold, spores, plant pollens such as tree, weed, and grass pollens. In other embodiments, the allergen is a food allergen. It will be appreciated that food allergies are atopic disorders that are mechanistically distinct from non-atopic disorders, such as celiac disease. Food allergies can be broadly classified into those that are IgE-mediated, those that are mediated by both IgE-dependent and IgE-independent pathways (mixed), and those that are not IgE-mediated. The immune mechanisms underlying food allergies are further described in, e.g., Wong et al., Nat Rev Immunol., 16 (12): 751-765 (2016). The immune complex may comprise an IgA immunoglobulin and any one or combination of food allergens. For example, the food allergen may be a peanut allergen, a tree nut allergen, a dairy allergen, a wheat allergen, a sesame allergen, a soy allergen, an egg allergen, a shellfish allergen, a meat allergen, and/or a corn allergen.

In some aspects, the composition desirably comprises a carrier, such as a pharmaceutically acceptable carrier. As used herein, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintrigrants (e.g., potato starch or sodium starch glycolate), polyethylene glycol, and the like. The compositions also can include stabilizers and preservatives. Examples of carriers, stabilizers and adjuvants have been described and are known in the art (see e.g., Martin, Remington's Pharmaceutical Sciences, 15th Ed., Mack Publ. Co., Easton, Pa. (1975)).

The choice of carrier will be determined in part by the particular immune complex used and method of administration. For example, the pharmaceutical composition may contain preservatives, such as, for example, methylparaben, propylparaben, sodium benzoate, and benzalkonium chloride. A mixture of two or more preservatives optionally may be used. In addition, buffering agents may be used in the composition. Suitable buffering agents include, for example, citric acid, sodium citrate, phosphoric acid, potassium phosphate, and various other acids and salts. A mixture of two or more buffering agents optionally may be used. Methods for preparing administrable (e.g., parenterally administrable) compositions are known to those skilled in the art and are described in more detail in, for example, Remington: The Science and Practice of Pharmacy, Lippincott Williams & Wilkins; 21st ed. (May 1, 2005).

In some embodiments, the composition can employ time-released, delayed release, and sustained release delivery systems such that the delivery of the composition occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician, and may be particularly suitable for certain embodiments.

The composition desirably comprises an “effective amount” of the immune complex, i.e., a dose or concentration of the immune complex which provokes a desired immune response (e.g., tolerance) in a recipient (e.g., a human). For example, the composition may comprise a therapeutically effective amount of the immune complex, as described above. Alternatively, the pharmacologic and/or physiologic effect may be prophylactic, i.e., the effect completely or partially prevents a disease or condition or symptom thereof. In this respect, the disclosed composition comprises a “prophylactically effective amount” of the immune complex. A “prophylactically effective amount” refers to an amount effective, at dosages and for periods of time necessary, to achieve a desired prophylactic result (e.g., prevention of an allergy or allergic reaction). For example, a composition comprising the immune complex may be administered to a subject (e.g., an infant) prior to development of allergy.

An effective amount can be administered in one or more administrations (e.g., via the same or different route) or applications and is not intended to be limited to a particular formulation or administration route. The composition comprising an effective amount of the IgA-allergen immune complex can be administered to a mammal (e.g., a human) using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection. In some embodiments, the composition is formulated for intranasal administration.

The disclosure provides compositions and methods for treating (e.g., prophylactically and/or therapeutically) a subject with an allergy or predisposed to experiencing allergy comprising providing an immune complex of the disclosure to the subject (e.g., thereby reducing and/or ameliorating Th2 type allergic immune responses and/or promoting mucosal tolerance toward the IgA targeted allergen in the subject). In some embodiments, a composition comprising immune complex is delivered enterally. In other embodiments, a composition comprising immune complex is delivered via pulmonary administration (e.g., via inhalation). In still further embodiments, a composition comprising immune complex is administered via two or more routes described herein.

In some embodiments, direct delivery and/or encapsulation of IgA immune complex via oral administration is used to promote mucosal tolerance (e.g., locally within the gut and/or at distant mucosal sites such as within the airway). Delivery of IgA immune complex may be independent of other compositions. In some embodiments, encapsulation (e.g., with biodegradable microparticles) may be used (e.g., to alleviate degradation of the IgA immune complex while passing through the low PH of the stomach). The disclosure is not limited to any particular encapsulation technology. In some embodiments, the encapsulation of the immune complex utilizes reagents such as polymer poly(ethylene glycol)-block-polycaprolactone (PEG-b-PCL) based nanoparticles or 1-palmitoyl-2-oleoyl-sn-glycero-3-phospho-(1′-rac-glycerol) (POPG).

In accordance with the disclosed method, the composition comprising the immune complex desirably is administered to a subject that has an established Th2 polarized immune response (e.g., at one or more mucosal sites) prior to administering the composition. It will be appreciated that there are two main subsets of T lymphocytes, distinguished by the presence of cell surface molecules known as CD4 and CD8. T lymphocytes expressing CD4 are also known as helper T cells, which are the most prolific cytokine producers. CD4+ T cells can be further subdivided into Th1 and Th2 cells, and the cytokines they produce are known as Th1-type cytokines and Th2-type cytokines. Th1-type cytokines (e.g., IFN-γ and/or tumor necrosis factor (TNF)) tend to produce the proinflammatory responses responsible for killing intracellular parasites and for perpetuating autoimmune responses. Excessive proinflammatory responses can lead to uncontrolled tissue damage, which may be counteracted by Th2-type cytokines. The Th2-type cytokines include interleukins 4 (IL-4), 5 (IL-5), and 13 (IL-13), which are associated with the promotion of IgE and eosinophilic responses in atopy, and also interleukin-10, which exhibits more of an anti-inflammatory response. Thus, in some embodiments, administering the composition comprising the IgA immune complex increases the expression of IL-10, TGF-β1, IFN-γ, IL-17, and/or CCL2 in the subject. In excess, Th2 responses will counteract the Th1-mediated microbicidal action. Ideally, humans produce a well-balanced Th1 and Th2 response. Th2 immune responses are characterized by the production of IgE antibodies and high levels of Th2 cytokines, and are associated with inadequate protection against some pathogens as well as cancer, colitis, asthma, and allergy. Th2 cells predominate in most patients with allergies and asthma and differentiate from uncommitted precursor T cells under the influence of IL-4. Th2 cells orchestrate allergic inflammation through the release of the Th2 cytokines IL-4, IL-5, IL-9, and IL-13.

In some embodiments, administration of a composition comprising the immune complex described herein results in modulation of an existing Th2 immune response in a subject. For example, the disclosed method provides the ability to redirect Th2-polarized immune responses in a subject (e.g., in a subject with Th2 mediated disease) toward a Th1-type immune response (e.g., skew toward a Th1 type immune response and/or generate a more balanced Th1/Th2 type immune response) via exposing the subject to the IgA immune complex. In some embodiments, administering an immune complex-containing composition to the subject increases the expression of Th1 type cytokines (e.g., IFN-γ and/or tumor necrosis factor (TNF)) in the subject. Thus, the disclosed method may be used to vaccinate individuals against allergies (e.g., peanut or other food allergies, respiratory allergies, etc.). In one embodiment, the invention provides a more effective benefit (e.g., more significantly reduced sign, symptom or cause of allergy, or a longer lived reduction of sign, symptom or condition of allergy) than the benefit attained with conventional allergy shots.

In some embodiments, administration a composition comprising the IgA immune complex described herein primes, enables, and/or enhances induction of both humoral (e.g., development of specific antibodies) and cellular (e.g., cytotoxic T lymphocyte) immune responses (e.g., thereby ameliorating signs, symptoms or conditions of allergic disease). Ideally, administering the IgA immune complex composition induces the development of regulatory T cells (Tregs) in the subject. The term “regulatory T cells (Tregs),” as used herein, refers to a specialized subpopulation of T cells that act to suppress immune responses, thereby maintaining homeostasis and self-tolerance. Tregs are able to inhibit T cell proliferation and cytokine production and play a critical role in preventing autoimmunity. Tregs can inhibit immune responses via suppression by inhibitory cytokines, suppression by cytolysis, suppression by metabolic disruption, or suppression by modulation of dendritic-cell (DC) maturation or function. With respect to inhibitory cytokines, interleukin-10 (IL-10), transforming growth factor-β (TGFβ) and IL-35 are key mediators of Treg cell function. Both mouse and human Treg cells have been shown to mediate cytolysis via granzyme A and/or granzyme B and perforin in vitro and in vivo.

Tregs are characterized by the expression of the master transcription factor forkhead box P3 (Foxp3). Although Foxp3 expression is widely used as a marker of the Treg lineage, recent data show that the Treg fate is determined by a multifactorial signaling pathway, involving cytokines, nuclear factors, and epigenetic modifications. Several subpopulations of human Treg cells have been identified based on expression levels of FOXP3 and CD45RA. Such subpopulations have been classified as naive/resting (CD45RA⁺FoxP3^{low}), effectors (CD45RA⁻FoxP3^{high}), and cytokine-producing (CD45RA⁻FoxP3^{low}). Naive Tregs arise from thymus with a fully demethylated FoxP3 locus. Effectors Tregs are the active population in vivo, while the cytokine-producing Tregs include those cells able to produce pro-inflammatory cytokines like IL-17 and IFN-γ but still able to suppress immune responses. Tregs are further described in, e.g., Kondělková et al., ACTA MEDICA (Hradec Králové) 2010; 53 (2): 73-77; Vignali et al., Nat Rev Immunol. 2008 July; 8 (7): 523-532. doi: 10.1038/nri2343; and Romano et al., Front. Immunol., 31 Jan. 2019; doi.org/10.3389/fimmu.2019.00043.

Furthermore, in some embodiments, compositions comprising an IgA immune complex induces (e.g., when administered to a subject) both systemic and mucosal immune responses (e.g., generates systemic and or mucosal immunity (e.g., thereby reducing or preventing signs, symptoms or conditions of allergic disease)). Thus, in some embodiments, administration of the disclosed composition results in protection against an exposure to one or a plurality of allergens and/or allergic substances (e.g., a food allergen). Ideally, the administration of the IgA immune complex-containing composition results in decreased hypersensitivity to the allergen in the subject upon subsequent exposure to the allergen.

The disclosed method can be performed in combination with other therapeutic methods to achieve a desired biological effect in a patient. Ideally, the disclosed method may include, or be performed in conjunction with, one or more therapeutic agents or regimens that ameliorate the symptoms and signs of an allergy and/or a Th2 polarized immune response at one or more mucosal sites. For example, the disclosed method may be performed in combination with (e.g., at the same time, via the same route as and/or in an escalating dose scheme similar to) one or more immunotherapeutic agents or treatment regimens to desensitize individuals to potential food allergens. Desensitizing immunotherapy is generally delivered sublingually, orally, or through the skin. Sublingual immunotherapy, or SLIT, involves administering a liquid extract of the allergen under the tongue, where it is held for several minutes. Daily allergen doses begin in the submilligram range and increase gradually over a period of days or weeks. The first double-blind, placebo-controlled trial of SLIT for food allergy was published in 2005 (Enrique et al., J. Allergy Clin. Immunol., 116:1073-1079 (2005)), and a large, multicenter, randomized, placebo-controlled, double-blind, crossover study in 2013 evaluated SLIT for peanut allergy (Fleischer et al., J. Allergy Clin. Immunol., 131:119-127 (2013)). In oral immunotherapy, or OIT, a low dose of allergen (in the milligram range) is ingested daily and the dose is gradually increased (e.g., every two weeks) over a period of several months. Because of the larger allergen doses that are used in OIT compared with other forms of immunotherapy, patients can often be desensitized not only to amounts of the allergen sufficient to avoid a life-threatening reaction due to accidental exposure, but also to the extent that they are able to consume gram amounts of allergenic foods. Epicutaneous immunotherapy, or EPIT, employs an adhesive containing microgram amounts of allergen to deliver antigen to the skin surface. This route of delivery seems to have fewer and less intense side effects than OIT, and some subjects may prefer wearing a skin patch to orally consuming the same food allergen each day.

In other embodiments, the disclosed method may be performed in conjunction with monoclonal antibody therapy. Several monoclonal antibodies have been developed to block the processes associated with allergic immune responses. For example, the monoclonal antibody omalizumab (XOLAIR®) binds to the Fc region of IgE antibodies, blocking IgE binding to FcERI and thus preventing the Fc receptor-mediated activation and degranulation of mast cells and basophils (Pennington et al., Nat Commun., 7:11610 (2016)). Omalizumab was originally approved for the treatment of allergic asthma, but has been tested in combination with OIT for the treatment of food allergies in a series of smaller studies (Nadeau et al., Clin. Immunol., 127:1622-1624 (2011); Schneider et al., J. Allergy Clin. Immunol., 132:1368-1374 (2013); Wood et al., J. Allergy Clin. Immunol., 137:1103-1110 (2016); and Begin et al., Allergy Asthma Clin. Immunol., 10:7 (2014)). Monoclonal antibodies that target upstream mediators of food allergy may also be used in the methods described herein. For example, monoclonal antibodies that bind to IL-5, such as mepolizumab (NUCALAR) and reslizumab (CINQAIR®), have been evaluated for treating eosinophilic oesophagitis (EoE), which can be triggered by milk allergens (Assa'ad et al., Gastroenterology, 141:1593-1604 (2011); and Spergel et al., J. Allergy Clin. Immunol., 129:456-463 (2012)). Therapeutic monoclonal antibodies may be administered in conjunction with immunotherapies as described above. Current and future potential treatments for food allergies are described in detail in, for example, Yu et al., Nat Rev Immunol., 16 (12): 751-765 (2016), any one or more of which may be used in combination with the compositions and methods disclosed herein.

The following example further illustrate the invention but, of course, should not be construed as in any way limiting its scope.

### EXAMPLES

The following materials and methods were used in the experiments described in the Examples.

### Mice

6-8 week-old BALB/c mice were obtained from The Jackson Laboratory (Bar Harbor, ME). DO11.10-Lky-IL-4GFP mice were donated by Dr. Simon Hogan, University of Michigan Medical School, Ann Arbor, Michigan. The mice were maintained in a clean barrier facility and were handled under an approved Institutional Animal Care and Use Committee protocols at University of Michigan animal facility.

### Reagents

Purified IgA from MOPC 315 cells (ECACC 85022106) which recognizes DNP-TNP substituted proteins was purchased from MP Biochemicals, USA. 2,4,6-trinitrophenyl hapten conjugated to ovalbumin protein (TNP-Ova) was purchased from Biosearch Technologies, USA.

### Ova Induced Intestinal Anaphylaxis

6-8 week-old mice were twice sensitized to OVA (50 μg of OVA/1 mg of alum) in sterile saline by intraperitoneal (i.p.) injection on day 0 and on day 14. During the second sensitization mice were intra-tracheally (i.t) treated with IgA+TNP-Ova; IgA alone; TNP-Ova alone; and saline respectively. Two weeks after the treatment, mice were subjected to repeated oral gavage (o.g.) challenges with OVA (50 mg Ova in 250 μl saline). Prior to each o.g. challenge, mice were deprived of food for 4-5 hours. Rectal temperatures were measured prior to challenge and then every 15 minutes up to 60 minutes. Diarrhea was assessed by visually monitoring mice for up to 60 minutes following o.g. challenge, and mice demonstrating profuse liquid stool were recorded as diarrhea-positive. Mice were considered allergic if they demonstrated symptoms of anaphylaxis (hypothermia>1.5° C. temperature loss and diarrhea) following the 4th challenge. Mice showing liquid stool were recorded as diarrhea-positive.

### Adoptive Transfer

T lymphocytes were collected from the spleens of DO11.10-Lky-IL-4GFP female mice. Spleens were placed in chilled RPMI 1640 (Bio-Whittaker, Walkersville, MD) and cells were isolated through a 40 μm nylon mesh filter. After red blood cell lysis, splenocytes were resuspended in RPMI 1640 and 5×10⁶ cells were plated and incubated with Ova peptides (100 μg/mL) in Th2 biased conditions in vitro (IL-4 (20 ng/ml; anti-IFN-γ (10 ug/mL); IL-2 (10 U/mL)) for 72-96 hours. CD4+KJ1.26+IL4-GFP+ cells were sorted using a cell sorter (BD Melody) and 1×10⁶ cells per mouse were adoptively transferred to the mice that were sensitized with Ova (50 μg of OVA/1 mg of alum, intraperitoneal) 7 days before the adoptive transfer. After 24 hours of the adoptive transfer, mice were intra-tracheally (i.t.) treated (day 8) with IgA+TNP-Ova; IgA alone; TNP-Ova alone; and saline respectively. On day 14, mice were subjected to repeated oral gavage (o.g.) challenges with OVA (50 mg Ova in 250 μl saline), and rectal temperature and diarrhea were recorded as described above. Flow cytometric analysis was performed on mesenteric lymph nodes to identify IL-4-GFP cells.

### Lymph Node Restimulation

Lung draining lymph nodes (LDLN), mesenteric lymph nodes, and Peyers patches were enzymatically digested using 1 mg/ml collagenase A (Roche) and 20 U/ml DNaseI (Sigma-Aldrich) in RPMI 1640 with 10% FCS for 45 minutes at 37° C. Tissues were further dispersed through an 18-gauge needle (1-ml syringe). RBCs were lysed, and samples were filtered through 100-μm nylon mesh. Cells (5×10⁵) from mLN cells were plated in 96-well plates and restimulated with Ova for 48 hours. IL-4, IL-5, IL-13, IL-17A, and IFN-γ levels in supernatants were measured with a Bio-Plex cytokine assay (Bio-Rad Laboratories).

### Quantitative PCR

Lung tissue was homogenized in TRIzol reagent and RNA was extracted using TRIzol reagent (Invitrogen, Carlsbad, CA). cDNA was synthesized using murine leukemia virus reverse transcriptase (Applied Biosystems, Foster City, CA) and incubated at 37° C. for 1 hour, followed by incubation at 95° C. for 10 minutes to stop the reaction. Real-time quantitative PCR (qPCR) was multiplexed using Taqman primers, with a FAM-conjugated probe to measure transcription of Il10, foxp3, Il4, Il5, Il13, Il17a, Ifng, and Ccl2. Fold change was quantified using 2-ΔΔ cycle threshold (CT) method normalized to 18s RNA and or naïve animals. Custom primers were designed to measure Muc5ac and Gob5 mRNA levels as described (Miller et al., 2004). All reactions were run on a ABI Prism 7500 Sequence Detection System (Applied Biosystems, Foster City, CA).

Serum IgE and mMCPT-1 Assay

Serum samples from blood drawn after cardiac puncture were analyzed using ELISA kits for OVA-specific IgE (MD Bioproducts, Oakdale, MN, USA), and mMCPT-1 (Invitrogen, Carlsbad, CA, USA). Total IgE (Bioscience, San Diego, CA, USA) and mMCPT-1 was performed for steady state analysis as per the manufacturer's instructions.

### Flow Cytometry

Lungs were removed, and single cells were isolated by enzymatic digestion with 2.5 mg/ml LIBERASE™ (Roche) and 20 U/ml DNaseI (Sigma, St. Louis, MO) in RPMI 1640 for 45 minutes at 37° C. or 1 mg/mL collagenase (Roche) and 20 U/ml DNaseI (Sigma, St. Louis, MO) in RPMI 1640+10% FCS for 60 minutes at 37° C. Tissues were further dispersed through an 18-gauge needle (5-ml syringe), RBCs were lysed and samples were filtered twice through 100-μm nylon mesh. Cells were resuspended in PBS. Live cells were identified using LIVE/DEAD Fixable Yellow Dead Cell Stain kit (Thermo Fisher Scientific, Waltham, MA), then washed and resuspended in PBS with 1% FCS. Fc receptors were blocked with purified anti-CD16/32 (clone 93; BioLegend, San Diego, CA). Surface markers were identified using antibodies (clones) against the following antigens (all from BioLegend): anti-CD3 (145-2C11), CD4 (GK1.5), CD8 (53-6.7), CD25 (3C7), CD69 (H1.2F3), CD19 (1D3/CD19), F4/80 (BM8), CD11c (N418), MHC II (M5/114.15.2), CD11b (M1/70), CD45 (30-F11), CD127 (A7R34), CD90 (30-H12), ST2 (DIH4), Gr-1 (RB6-8C5), B220 (RA3-6B2), and Ter119 (Ter-119). For innate lymphoid cell staining, lineage markers were anti-CD3, CD11b, B220, Gr-1, and TER119. ILC2: CD45+/Lin−/CD90+/ST2+. Data was collected using a NovoCyte flow cytometer (ACEA Bioscience, Inc. San Diego, California). Data analysis was performed using FlowJo software (Tree Star, Oregon, USA).

### Lamina Propria Mononuclear Isolation

Ileal loop tissue was opened longitudinally to remove Peyer's patches and mesenteric vessels, and LP-enriched fraction was enzymatically dissociated at 37° C. under mechanical agitation as described by Luissint et al., 2019. Briefly, mucus was removed by washing tissue in PBS-supplemented with 2% FBS and 5 mM DTT (Fisher BioReagents) for 20 minutes. The IEC lining was removed by three consecutive washings in chelation buffer (PBS-containing 2% FBS and 5 mM EDTA) for 10 minutes. Tissue was minced and digested in HBSS+ supplemented with 10 mM HEPES, LIBERASE™ (37.5 U/mL; Roche Applied Science, Indianapolis, IN) and DNase I (300 Kuntz units/mL) for 30 minutes. Cell suspension was filtered, washed in a solution of PBS-supplemented with 10% FBS and 2 mM EDTA, and cell numbers determined, and were stained for flow cytometry analysis.

### Histological Examination

Serial 6-μm sections were obtained from a paraffin-embedded, 10% formalin-fixed left lungs, and small intestines stained with H&E. Five sections were analyzed per mouse, with two lung/small intestine slices per section per mouse to select representative slides. PAS staining was performed to identify mucus in the airways. Chloro-acetate esterase staining was performed to identify mast cells in the small intestines.

### Statistical Analysis

Data were analyzed by Prism 7 (GraphPad Software). Data presented are mean values±SEM. Comparison of two groups was performed with an unpaired, two-tailed Student's t-test. Comparison of three or more groups was analyzed by one-way ANOVA, followed by two-tailed Student's t-test for individual comparisons. A p-value<0.05 was considered significant.

### Example 1

This example demonstrates that administration of IgA immune complexes into the airway inhibits allergic asthma responses in mice.

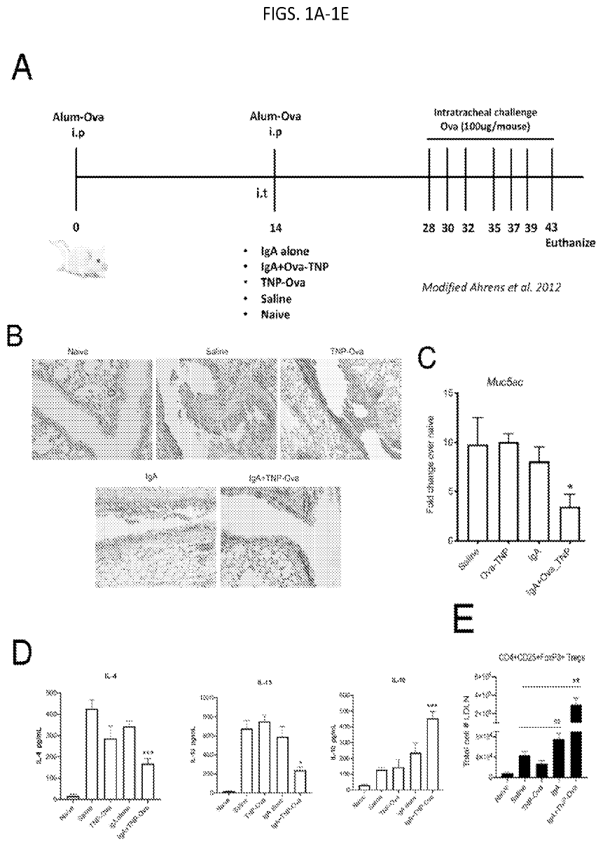

Examination of the role of IgA on regulation of immune responses has not been resolved with previous studies. Most of the support for IgA having an important role in tolerogenic and inhibitory responses has been circumstantial and related to deficiencies or correlated to IgA levels. It was examined whether antigen specific IgA with or without antigen would alter an ongoing allergic immune response. To this end, a model of allergic airway response with 2 alum-ovalbumin systemic sensitizations was employed to initiate a strong Th2 response. At the time of the 2^{nd} intraperitoneal alum ovalbumin sensitization the animals were given an intratracheal supplementation of a TNP-specific IgA, with or without TNP-Ovalbumin. The latter immune complex allowed IgA to induce a specific response, similar to what would happen at the mucosal surface during antigen exposure. Other controls included TNP-ova only and saline (vehicle). Two weeks after the 2nd ovalbumin alum-ova challenge the animals were given 7 ovalbumin challenges into the airway over a 2-week period. Twenty-four hours after the final ovalbumin airway challenge the animals were examined for evidence of allergic airways disease (FIG. 1A). Examination of the histopathology stained with PAS suggests that the IgA-TNP-ova immune complex treated animals have reduced mucus staining the airways compared to all the other groups (FIG. 1B), which was confirmed by examining the expression of muc5b, the primary mucin expressed in the lung by goblet cells (FIG. 1C). Restimulation of lung draining lymph node cells demonstrated that the IgA/TNP-ova treatment group had significant decreases in Th2 cytokines, IL-4, IL-5, IL-13, with an increase in IL-10 (FIG. 1D). Furthermore, flow cytometry analysis of the lymph node cells demonstrated a significant increase in CD4+CD25+Foxp3+ T cells only in the IgA/TNP-ova treatment group with a nearly 10-fold increase compared to saline control treated allergic animals (FIG. 1E). Thus, the local mucosal application of IgA/TNP-ova in animals with systemic allergic responses to ova protected animals to local allergen challenges with a corresponding increase in Treg cells.

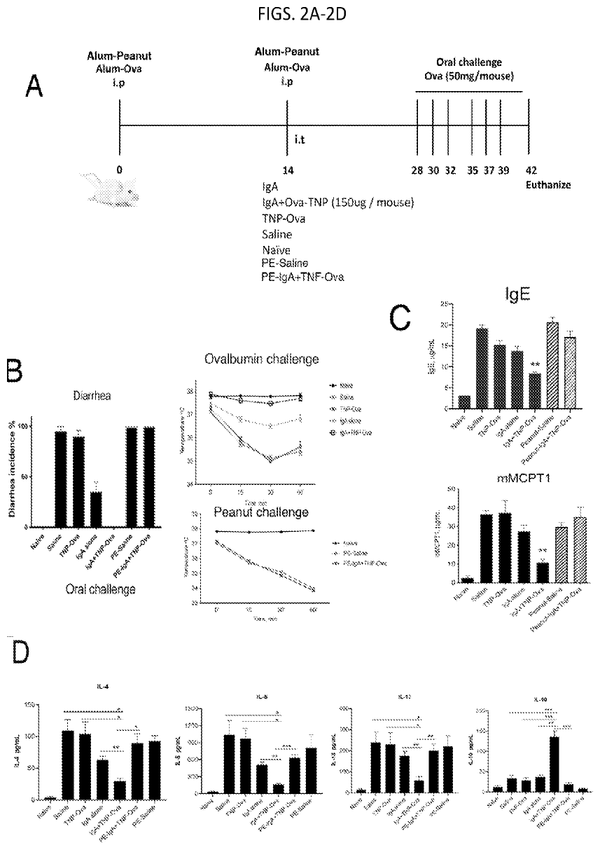

The reduction of an established immune response in sensitized mice with IgA-immune complex to a mucosal airway challenge above, indicated an association with development Treg cell responses. In order to understand if the response was local or if the airway applied IgA immune complex had a systemic effect, studies were designed to investigate intestinal food allergy using a similar strategy. Using a systemic sensitization model to induce an allergic response with alum-ovalbumin given by IP, similar to the airway model, a similar IgA immune complex application into the airway was followed (FIG. 2A). In addition, mice were immunized with peanut allergen (Greer) precipitated with Alum to check for allergen specific responses (FIG. 2A). Just prior to a 2^{nd} alum-ova sensitization the animals were given an airway (IT) immune complex application or appropriate controls. The intragastric ovalbumin food challenges were started 2 weeks later with 4 oral gavages as indicated. The peanut-Alum sensitized mice were given PE-Saline or PE-IgA+TNF-Ova to check whether the altered ovalbumin response with IgA immune complex was allergen specific.

A primary outcome of the responses was the development of diarrhea within 30 minutes of the oral ovalbumin challenge. Examining diarrhea in the different groups demonstrated that while TNP-ova given into the airway on its own did not alter the ongoing allergic responses, the IgA-TNP-ova immune complex did alleviate the development of the diarrhea responses (FIG. 2B). In contrast, the peanut-alum sensitized mice treated with IgA-TNP-ova immune complex showed no reduction in diarrhea compared to peanut control mice (FIG. 2B). The reduction in temperature, another indication of anaphylaxis was significantly reduced when animals were challenged, but not with those animals treated with the IgA immune complex. To further examine the development of the allergic response, IgE was examined along with mast cell derived MCPt (FIG. 2C) in serum at 60 minutes post-oral challenge. Both IgE and MCPt were significantly reduced only in the animals exposed to the IgA immune complex into the airway in animals challenged with ovalbumin. The overall immune responses were also examined by re-stimulating gut draining lymph node cells with ovalbumin. The resulting supernatant demonstrated a significant decrease in IL-4 and IL-13, with increased IL-10 in the IgA immune complex treated animals (FIG. 2E). In contrast, none of the immune parameters, including IgE, MCPT1, or cytokines were altered in peanut-sensitized and challenged mice by the IgA-TNP-ova immune complex (FIGS. 2C-E). Together, these data demonstrate a significant alteration induced by an airway IgA/TNP-ova immune complex exposure during a systemic allergic response protected animals from severe Th2-induced oral antigen responses, and it was allergen specific.

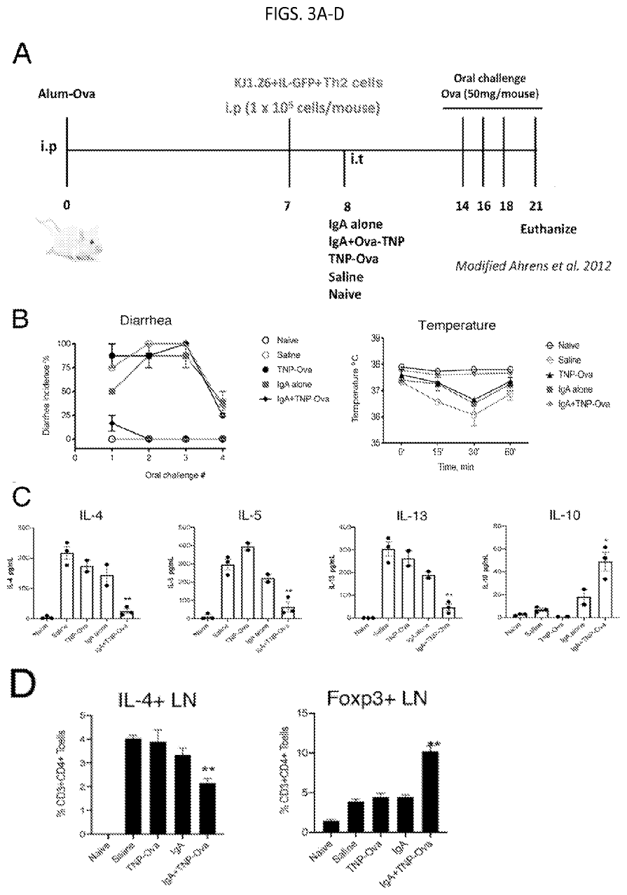

In order to better understand how the IgA immune complex was altering the allergic response in sensitized mice, a model of T cell transfer was utilized with Th2 skewed DO11 ova-specific TCR transgenic T cells. This model was set up by isolation of GFP IL-4 reporter DO11 naïve splenocytes. The splenocytes from naïve DO11 mice were skewed in vitro with rIL-4, anti-IFN, rIL-2, and TCR activation (see Material and Methods) and sorted for GFP expression, which indicated IL-4 production. Since the model used an already skewed Th cell subset, the immunization protocol was modified to a single alum-ova sensitization followed by the treatment of animals at day 7 with sorted IL-4 producing skewed T cells by IP injection into the Balb/c mice, followed by the IgA/TNP-ova immune complex 24 hours later into the airway (FIG. 3A). On day 14 challenge by oral gavage began, and on the 4th challenge the animals were examined for the development of diarrhea. The IgA/TNP-ova composition nearly completely protected the mice from severe diarrhea and from decreased temperature both indications of anaphylaxis (FIG. 3B). When draining lymph nodes were restimulated ex vivo, a significant reduction in IL-4, IL-5, and IL-13 were observed with a significant increase in IL-10 (FIG. 3C). When flow cytometry was performed to identify the GFP+IL-4+ T cells, a significant reduction was found in the IgA/TNP-ova treated group, with a corresponding increase in the Foxp3+ Treg cells (FIG. 3D). Importantly, none of the Foxp3+ T cells were IL-4+, indicating that they were derived from a naïve host T cell and not from a previously Th2 skewed cell.

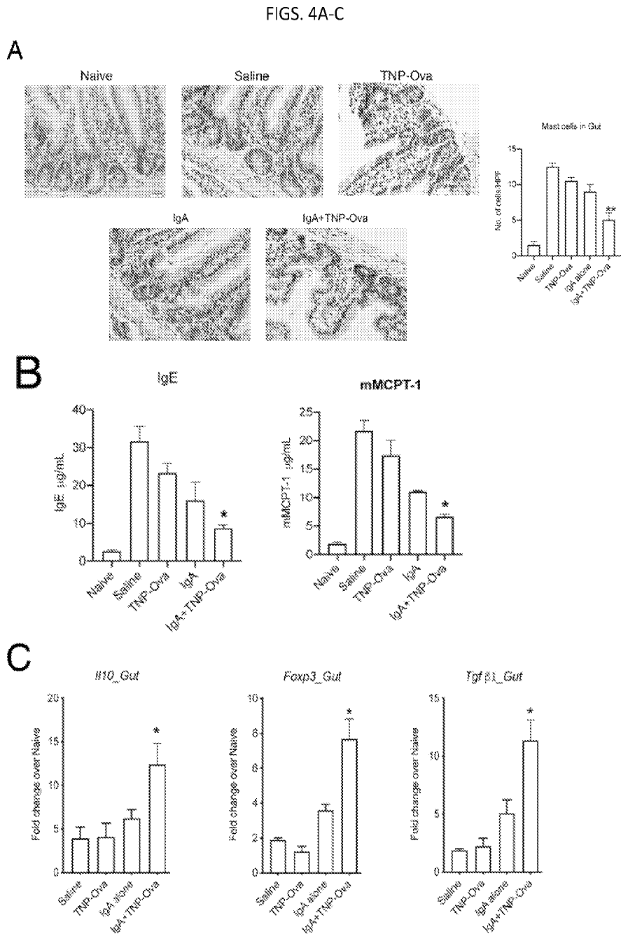

In order to further describe the pathogenesis of disease, studies were performed to examine the number of mast cells that accumulated in the small intestine, as they appear to correlate to the severity of the anaphylactic disease response. The IgA/TNP-ova immune complex treated animals showed a significant decrease in mast cells in the small intestine (FIG. 4A). The levels of IgE and mMCPt-1 in serum were also significantly reduced only in the IgA/TNP-ova immune complex treated animals (FIG. 4B), further demonstrating reduced Th2 and mast cell activation. Furthermore, when expression levels of IL-10, TGF-β, and foxp3 were examined in the gut tissue, a significant upregulation of these genes were observed (FIG. 4C). Thus, the ability to regulate Th2 immune responses corresponds to the development of increased mast cell numbers in the small intestine of allergic mice and can be modulated by a mucosal treatment with allergen specific IgA immune complexes.

One potential mechanism of how the IgA immune complex facilitates the altered response would be via differential activation of antigen presenting cells (APC), especially dendritic cells (DC). To investigate, bone marrow derived DC were grown in GM-CSF for 6-7 days and exposed to IgA immune complex or the appropriate controls. After overnight incubation the DC were assessed for innate cytokine expression, IL-10 and TGFβ, by quantitative PCR analyses (FIG. 6). The data demonstrate that the IgA immune complex induced significantly increased mRNA expression of TGFβ (FIG. 6A) and IL-10 (FIG. 6B), while IgA alone increased them to a much lesser extent. To better understand if the immune complex activation alters the ability of the DC to elicit an antigen specific primary immune response DO11 ovalbumin peptide transgenic TCR naïve splenic CD4 T cells were used. After an overnight incubation with IgA immune complex or appropriate controls, the DC were washed and replated with DO11 naïve CD4 T cells at a 1:10 ratio with new ovalbumin for processing and presentation to the T cells. The data in FIG. 6C indicate that while there was a substantial production of all cytokines in response to ovalbumin compared to the only DC control (given no ovalbumin), the IgA immune complex pre-incubated DC had significantly decreased IL-4, IL-13 and IL-17, but increased IL-10 and IFNγ in supernatants after a 48 hr incubation. Altogether, these data indicate that the IgA immune complex induces regulatory cytokines in DC that correspond to altered T cell responses.

### Example 2

### This Example Describes Oral Administration of IgA Immune Complexes

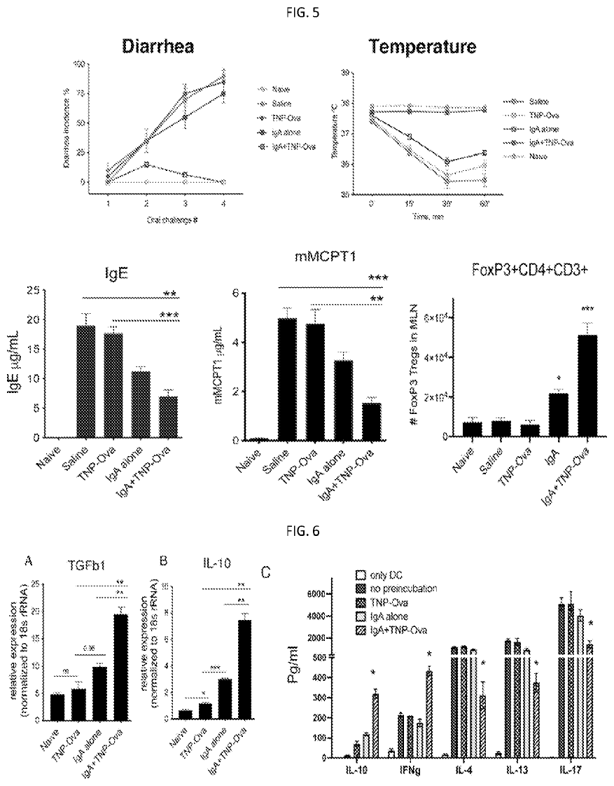

The IgA immune complex with TNP-ovalbumin was given by oral gavage in systemically sensitized mice to promote a similar regulatory effect in anaphylactic disease. Systemically sensitized animals were given the IgA: TNP-ovalbumin immune complex, or appropriate control treatments, by oral gavage at day 14 prior to the second Alum-ovalbumin IP sensitization. As above in FIG. 2, on day 28 of the protocol, 14 days after the 2^{nd} alum-ovalbumin sensitization, oral gavage challenges were given with ovalbumin. After the final challenge animals were monitored for diarrhea and temperature changes and showed that only those animals given the IgA IC by oral gavage were protected from anaphylactic disease as assessed by diarrhea incidence and temperature change (FIG. 5). In addition, IgE and MCPT1 were also significantly decreased in only the IgA-TNP-ova IC treated animals. The Treg cells were increased in lymph nodes of animals treated with IgA IC by ˜5 fold, whereas IgA only also showed a more modest increase in Treg cells but was not sufficient for altering severe anaphylactic ova induced disease. Thus, the IgA IC to ovalbumin can be provided at both the airway and gut mucosal surface to induce a regulatory response to protect against severe allergen-induced sequelae.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising”, “having,” “including,” and “containing are to be construed as open-ended terms (i.e., meaning including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inhibiting an allergic reaction in a subject, which method comprises administering to the subject a composition comprising an immune complex and a pharmaceutically acceptable carrier, wherein the immune complex comprises an allergen bound to an IgA immunoglobulin specific for the allergen, and wherein the subject has an established Th2 polarized immune response at one or more mucosal sites prior to administering the composition.

2. The method of claim 1, wherein the allergen is a food allergen, an aeroallergen, an animal product, a drug, insect venom, or latex.

3. The method of claim 2, wherein the allergen is a food allergen selected from a peanut allergen, a tree nut allergen, a dairy allergen, a wheat allergen, a soy allergen, an egg allergen, a shellfish allergen, a meat allergen, a sesame allergen, and a corn allergen.

4. The method of any one of claims 1-3, wherein the subject is a human.

5. The method of any one of claims 1-4, wherein administering the composition increases the expression of IL-10, TGF-β1, IFN-γ, IL-17, and/or CCL2 in the subject.

6. The method of any one of claims 1-5, wherein administering the composition induces the development of regulatory T cells (Tregs) in the subject.

7. The method of any one of claims 1-6, wherein administering the composition to the subject increases the expression of Th1 type cytokines in the subject.

8. The method of any one of claims 1-7, wherein the composition is administered to the subject intranasally.

9. The method of any one of claims 1-8, wherein hypersensitivity to the allergen is decreased in the subject upon subsequent exposure to the allergen.

10. Use of a composition comprising a pharmaceutically acceptable carrier and an immune complex comprising an allergen bound to an IgA immunoglobulin, or antigen-binding fragment thereof, for inhibiting an allergic reaction to a food allergen in a subject, wherein the IgA immunoglobulin is specific for the allergen.

11. The use of claim 10, wherein the allergen is a food allergen, an aeroallergen, an animal product, a drug, insect venom, or latex.

12. The use of claim 11, wherein the allergen is a food allergen selected from a peanut allergen, a tree nut allergen, a dairy allergen, a wheat allergen, a soy allergen, an egg allergen, a shellfish allergen, a meat allergen, and a corn allergen.

13. The use of any one of claims 10-12, wherein the composition is formulated for enteral administration.

14. The use of any one of claims 10-13, wherein the composition is formulated for oral administration.

15. The use of any one of claims 10-12, wherein the composition is formulated for mucosal administration.
